(19) United States
(12) Patent Application Publication
(10) Pub. No.: US 2026/0283560 A1
(43) Pub. Date: Sep. 24, 2026

(54) DEEP LEARNING-BASED PHOTOPLETHYSMOGRAPHY MODEL FOR CARDIOVASCULAR RISK PREDICTION

(71) Applicant: GOOGLE LLC, Mountain View, CA (US)

(72) Inventors: Wei-Hung WENG, San Franisco, CA (US); Sebastien Jean Jean-Paul BAUR, Toulouse, Occitanie (FR); Mayank DASWANI, London (GB); Diego ARDILA, Oakland, CA (US); Yun Irwin HERRMANN, Mountain View, CA (US); Christina Whetin CHEN, San Francisco, CA (US); Sujay Shivanand KAKARMATH, Reading, PA (US)

(21) Appl. No.: 19/477,638

(22) PCT Filed: Apr. 23, 2024
(86) PCT No.: PCT/US2024/025856

§ 371 Date: Oct. 22, 2025

Related U.S. Application Data
(60) Provisional application No. 63/497,911, filed on Apr. 24, 2023.

(51) Int. Cl. A61B 5/00; A61B 5/024

(52) U.S. Cl. CPC A61B 5/7267; A61B 5/02416; A61B 5/7275

(57) ABSTRACT

Accurate prediction of the risk of cardiovascular disease (CVD) can lead to significantly improved health outcomes by prompting earlier, more effective, and lower-cost interventions (like lifestyle and diet changes). Available long-term predictive tests for CVD risk include costly, clinically intensive diagnostics that include the efforts of highly trained clinical or laboratory staff like blood or other fluid draws and related laboratory testing, ECG, sphygmomanometer-based blood pressure measurement, or other tests. Such testing is not available in resource-constrained healthcare settings. Embodiments are provided that use a trained machine learning model to generate, from photoplethysmographic (PPG) signals, features that can be used, in combination with readily available information like age and sex and PPG-derived heart rate, an estimate of CVD risk. PPG sensors are readily available, low-cost, and are able to be used without training, allowing the embodiments herein to be used to predict CVD risk even in low-resource healthcare systems.

### CROSS-REFERENCE TO RELATED APPLICATIONS

The present application is a non-provisional patent application claiming priority to U.S. Provisional Patent Application No. 63/497,911, filed Apr. 24, 2023, the contents of which are hereby incorporated by reference.

### BACKGROUND

It is desirable to detect, as early as is feasible, the progression of cardiovascular disease (or other chronic, progressive health conditions like diabetes, hypertension, etc.). This can allow for early interventions (e.g., diet and lifestyle changes, preventive pharmaceuticals) to reduce the severity of the disease or even to prevent its development altogether, more efficient allocation of treatment or diagnostic resources (e.g., performing a more expensive and more conclusive diagnostic, increasing the frequency of future diagnostics or other interventions), or other benefits. The current standard for predicting the current presence, or likelihood of future development, of cardiovascular disease includes a patient traveling to a healthcare facility (e.g., a general practitioner's office) and undergoing diagnostic testing, including measurement of blood pressure (and optionally additional laboratory measures, e.g., blood draws) by a trained healthcare worker using additional diagnostic equipment. However, this method is costly with respect to the time and resources needed.

### SUMMARY

In a first aspect, a method for training a composite model to predict cardiovascular disease risk is provided that includes: (i) training a machine learning model of the composite model to output, based on an input photoplethysmographic waveform, a first set of features that are representative of the photoplethysmographic waveform; (ii) determining, based on the photoplethysmographic waveform, a heart rate; (iii) determining, based on the first set of features, a mapping of the composite model to project the first set of features to a second set of features, wherein the second set of features includes fewer features than the first set of features; (iv) determining a set of model coefficients of the composite model to predict, based on the second set of features, the heart rate, and a set of demographic information, a cardiovascular disease risk score; and (v) outputting an indication of the machine learning model, mapping, and set of model coefficients of the composite model.

In a second aspect, a method is provided that includes: (i) obtaining a photoplethysmographic waveform for a subject; (ii) determining, based on the photoplethysmographic waveform, a heart rate for the subject; (iii) applying the photoplethysmographic waveform to a machine learning model to generate a set of features for the subject; and (iv) applying the heart rate, the set of features, and a set of demographic information for the subject to a statistical model to determine a cardiovascular disease risk for the subject.

In a third aspect, a method for training a model to predict an output from a time series input is provided that includes: (i) obtaining a training example, wherein the training example includes a time series input; (ii) generating an augmented training example that includes an augmented time series input by: (a) generating a random time-varying time shift; and (b) applying the time-varying time shift to the time series to generate the augmented time series; and (iii) training the model based on a training dataset that includes the training example and the augmented training example.

In another aspect, a non-transitory computer readable medium is provided having stored thereon program instructions executable by at least one processor to cause the at least one processor to perform any of the above methods.

In another aspect a system is provided that includes: (i) at least one processor, and (ii) a non-transitory computer-readable medium, having stored therein instructions executable by the at least one processor to cause the system to perform any of the above methods.

These as well as other aspects, advantages, and alternatives will become apparent to those of ordinary skill in the art by reading the following detailed description with reference where appropriate to the accompanying drawings. Further, it should be understood that the description provided in this summary section and elsewhere in this document is intended to illustrate the claimed subject matter by way of example and not by way of limitation

### BRIEF DESCRIPTION OF THE FIGURES

FIG. 1 illustrates a flowchart of an example machine learning model training and inference process.

FIG. 2 illustrates aspects of an example system.

FIG. 3 illustrates a flowchart of an example method.

FIG. 4 illustrates a flowchart of an example method.

FIG. 5 illustrates a flowchart of an example method.

FIG. 6 depicts experimental results.

FIG. 7 depicts experimental results.

FIG. 8 depicts experimental results.

### DETAILED DESCRIPTION

### I. Overview

It would be beneficial to develop an alternative assay that could detect the likelihood of development of cardiovascular disease of similar quality (e.g., C-statistic, specificity, sensitivity) as existing in-office methods while relying on less-expensive equipment and/or while requiring minimal trained healthcare worker time to perform.

The systems and methods described herein use the photoplethysmographic waveform (PPG), in combination with readily available demographic information, to accurately predict the likelihood that a patient (subject) will develop cardiovascular disease. The PPG waveform represents the variation over time of the volume of blood in the capillary bed of the skin, and can be measured in a variety of ways (e.g., by illuminating the skin and detecting backscattered light, by illuminating the skin and detecting light transmitted through a finger or other protruding body part, by detecting a color of the skin from a distance over time) using a variety of types of equipment (e.g., finger-mounted pulse oximetry devices, pulse oximetry-sensing watches, pulse oximetry sensors in smartphones, smartphone cameras, other optical sensors in watches or smartphones).

The systems and methods described herein apply such PPG waveform data to a machine learning model (e.g., a ResNet18 model or other variety of deep learning model) to generate a set of output features that are representative of the PPG waveform. These features are then applied, in combination with a heart rate determined from the PPG waveform and demographic information (e.g., at least one of sex, age, and smoking status (whether the patient has ever been a smoker, and optionally all of these), and optionally BMI or other additional information), to a statistical model (e.g., a Cox proportional hazards model) to predict a score that is indicative of the likelihood that the patient will develop cardiovascular disease within a specified time period (e.g., ten years). The model-predicted output features are related to specific features of the PPG waveform morphology and are independent of heart rate.

The features that are representative of the PPG waveform and that are applied to the statistical model could be directly output from the machine learning model, or could be the result of projecting the model output into a lower-dimensional set of features. For example, a set of principal component analysis (PCA) eigenvectors or some other dimensionality-reduction method (e.g., independent component analysis, PCA using a nonlinear kernel, a support vector machine, an isometric mapping, multidimensional scaling, linear discriminant analysis, factor analysis, singular value decomposition, non-negative matrix factorization) could be used to reduce the number p of “first” features output from the model (e.g., 512 or more features, e.g. independently varying features) to a smaller number l of “second” features (e.g., five features), where l is less than p.

The eigenvectors may be selected based on a n×p matrix derived from the first features for PPG waveforms for a plurality of subjects, and denoted X where the number of rows n is an integer and the number of columns p is an integer which is the number of first features. For example, n may be a number of human subjects (for whom a PPG waveform is available), each row may correspond to a different subject, and each value of each element of the matrix may be the output of the machine learning model for the corresponding feature when a PPG waveform captured by a measurement of the corresponding subject is input to the machine laming model.

The calculation of the eigenvectors may be done by a standard method. For example, a number l of p-component orthogonal unit-length eigenvectors {ωₖ} for k=l, . . . l, may be successively selected such that each corresponding n-component vector tₖ≡Xωₖ has maximum variance. Once this is done, a dimensionality-reduction process may be applied to set of first features output by the machine learning model based on a PPG waveform for a specific process, by a linear projection based on the eigenvectors of the composite model, e.g. by obtaining the corresponding dot products of a p-component vector comprising the p first features respectively with each of the l eigenvectors, to give the l respective second features.

The trained machine learning model could be trained in a variety of ways using a variety of training data. For example, the machine learning model could be trained to predict, from input PPG waveforms, human-interpretable features determined from the PPG waveforms (e.g., pulse wave reflection index, peak to peak time, pulse wave peak position, pulse wave notch position, pulse wave shoulder position, whether a dicrotic notch is present, and/or an arterial stiffness index; here the peaks may be in pressure or in any other parameter indicated by the PPG waveform which varies during the cardiac cycle, e.g. with a single peak in each cycle) or some other set of available information that is relevant to the condition(s) or event(s) to be predicted for the individuals from whom the PPG waveforms had been detected (e.g., sex, age, BMI, hypertension status, hba1c, total cholesterol, systolic blood pressure, previous experience of at least one previous major adverse cardiovascular event, and PPG dicrotic notch presence). This could include training the machine learning model in combination with an output model (which could itself be a machine learning model) such that the output of the machine learning model is applied as an input to the output model, which then generates predictions of the target features (e.g., human-interpretable features of the input PPG waveform, a set of physiological data like age, sex, BMI, etc.). Additionally or alternatively, the machine learning model could be trained in a self-supervised manner (e.g., as part of an encoder-decoder pair), semi-supervised manner, or fully supervised manner to generate feature vectors that represent useful latent features in the set of PPG waveforms used as training data.

Training data used to train the machine learning model (and/or to determine coefficients of the statistical model that receives the output of the machine learning model) could include stored sets of PPG waveforms and associated demographic data (e.g., at least one of age, sex, smoking status, BMI) along with data about whether the corresponding individuals were diagnosed with cardiovascular disease (e.g., a date of diagnosis and/or experienced a myocardial infarction, stroke, cardiovascular-related death, or other event relative to a date of measurement of the PPG waveform and other demographic data, a severity of the cardiovascular disease suffered by the patient) and/or experienced some other cardiovascular event (e.g., myocardial infarction, stroke, cardiovascular-related death) such that they are in a higher-risk group. The amount of such data needed to train the machine learning model could be reduced by augmenting the available data. This could include using a process to generate additional PPG waveforms from the existing PPG waveforms by simulating random (e.g., pseudorandom) variations in the ‘rate of playback’ of the existing PPG waveforms, or otherwise imposing random time-varying time-shifts to the existing PPG waveforms. This could include generating a random time-varying playback speed (e.g., pseudo-randomly generated normally-distributed playback speed) and then using that playback speed to generate a corresponding time-varying time-shift (e.g., via a running sum) to be applied to the PPG waveform in order to simulate “playing back” the PPG according to the time-varying playback speed. Such a data augmentation method (which may be referred to as “Brownian tape speed augmentation”) could be applied in a variety of applications to a variety of training data, e.g., to training data that represents band-limited time-series data or other data whose essential features will not be abolished by the application of random (e.g., pseudo-random) time-shifts thereto.

Thus, in one form of the disclosure, a “composite model” is proposed comprising a machine learning model for processing an input photoplethysmographic waveform (e.g. captured from a certain human subject) to generate a first set of features representative of the waveform, optionally a dimensionality-reduction process (e.g. as explained above based on a plurality of eigenvectors associated with the composite model, such as determined based on a PCA analysis) to generate a second set of features from the first set, and a further model defined by a set of model coefficients designed to process the second set of features (or, in the absence of the dimensionality reduction model, the first set of features), and typically further data such as (i) data derived from the input photoplethysmographic waveform (i.e. derived other than via a machine learning model) such as a heart rate for the subject and/or (ii) data (e.g. demographic data) characterizing the subject, to output a cardiovascular disease risk score.

Optionally, based on the cardiovascular disease risk score, corresponding information may be output (e.g. if the method is carried out by a computer which is a piece of user equipment, the information may be output to the user using a screen of that user equipment). For example, the information may be a warning if the risk score is above a threshold. The warning may be in the form of a message to consult a health specialist. Alternatively or additionally, based on the cardiovascular disease risk score, it may be determined whether to administer a drug and/or apply a treatment to the subject, and the method may include administering that drug and/or that treatment. For example, if the risk score is above a certain threshold, the system may issue an instruction to administer to the subject a dose of a drug (e.g. a drug previously prescribed for the patient) and/or a treatment. Thus, for example, the present system and/or methods may recognize an emergency situation and trigger actions (e.g. actions pre-specified by a medical professional) appropriate for that situation.

The systems and methods described herein provide a variety of benefits. For example, since heart rate is represented by the information in the PPG waveform (e.g., as the reciprocal of the average inter-pulse interval), it is possible to train a machine learning model to generate heart rate as an output in addition to the other features, with this model-generated heart rate then provided as an input to the statistical model. However, such a machine learning model would require additional parameters (increasing computational cost and memory requirements at inference time, in additional to increasing the storage requirements to store a representation of the model), and would also require additional training examples and additional training time to train accurately. In contrast, the systems and methods described herein avoid such costs by determining the heart rate from the PPG waveform directly and to be trained using less training data. For example, by using a programmed algorithm rather than a trained adaptive system, e.g., a heuristic algorithm that includes identifying peaks within the PPG waveform (e.g. as peak pressure values) and then determining the reciprocal of the average inter-peak interval), allowing the machine learning model to be smaller (thus reducing memory and cycle requirements at inference time).

The systems and methods for determining a cardiovascular disease risk score or likelihood based, in part, on PPG waveforms are also improved relative to alternative methods that rely on the detection of blood pressure. Blood pressure detection requires expensive, highly-calibrated instruments and expensive, highly-trained medical professional time; alternatively, less expensive automated blood pressure measurement cuffs are less accurate and require significant amounts of power and time to measure blood pressure. Such instruments also include many mechanical components. In contrast, PPG can be measured quickly and a much lower-power manner using readily available, low-cost optical components (e.g., a light emitter and a photodiode, a CCD camera or other camera element, etc.). Such optical detection includes no (or few) moving parts, and so also exhibits improved reliability when compared to automated blood pressure detection. Further, the PPG waveform can be detected remotely, in a non-contact manner (e.g., by imaging the color of the face or other exposed skin from a distance in order to determine the PPG waveform), whereas blood pressure can only be measured via some degree of direct contact with the body.

Note that the systems and methods herein for the use of PPG waveforms and related demographic information to predict risk of cardiovascular disease can also be used to predict a risk score related to a variety of other progressive chronic diseases or disorders and/or medical events related thereto. For example, the systems and method escribed herein could be modified to predict a risk score relating to the likelihood of developing diabetes or hypertension within a specified time period. Additionally or alternatively, such systems and methods could be used to determine whether a patient is likely to be hospitalized, experience a cardiovascular event (e.g., heart attack, stroke), be prescribed a drug (e.g., a blood pressure drug, a heart disease drug, a diabetes drug), receive a treatment (e.g., an angioplasty, installation of a stent), or experience some other medical event or activity. Additionally or alternatively, the systems and methods herein may be used, in the case of the other progressive chronic diseases or disorders, to cause the display or a warning if a risk score is above a threshold, and/or determine a drug and/or a treatment to apply to a subject, and the method may include administering that drug and/or treatment.

### II. Example Machine Learning Models and Training Thereof

A machine learning model as described herein may include, but is not limited to: an artificial neural network (e.g., Transformers, layered models wherein each layer includes two or more sub-layers one or more of which could include artificial neural networks, convolutional neural networks, a recurrent neural network, a Bayesian network, a hidden Markov model, a Markov decision process, a logistic regression function, a support vector machine, a suitable statistical machine learning algorithm, and/or a heuristic machine learning system), a support vector machine, a regression tree, an ensemble of regression trees (also referred to as a regression forest), a decision tree, an ensemble of decision trees (also referred to as a decision forest), or some other machine learning model architecture or combination of architectures.

An artificial neural network (ANN) could be configured in a variety of ways. For example, the ANN could include two or more layers, could include units having linear, logarithmic, or otherwise-specified output functions, could include fully or otherwise-connected neurons, could include recurrent and/or feed-forward connections between neurons in different layers, could include filters or other elements to process input information and/or information passing between layers, or could be configured in some other way to facilitate the processing of input sequences, sets of embedding vectors representing input sequences, downstream vectors and/or set of vector determined by the operation of one or more layers or sublayers of a multi-layer model, and/or individual vectors (e.g., embedding vectors representing tokens of an input sequence, downstream vectors representing the processing of such embedding vectors by one or more layers or sublayers of a multi-layer model).

An ANN could include one or more filters that could be applied to the input and the outputs of such filters could then be applied to the inputs of one or more neurons of the ANN. For example, such an ANN could be or could include a convolutional neural network (CNN). Convolutional neural networks are a variety of ANNs that are configured to facilitate ANN-based classification or other processing based on images or other large-dimensional inputs whose elements are organized within two or more dimensions. The organization of the ANN along these dimensions may be related to some structure in the input structure (e.g., as relative location within the one-dimensional space of sequence of tokens can be related to similarity or relevance between tokens of the sequence).

In example embodiments, a CNN includes at least one two-dimensional (or higher-dimensional) filter that is applied to an input; the filtered input is then applied to neurons of the CNN (e.g., of a convolutional layer of the CNN). The convolution of such a filter and an input could represent the color values of a pixel or a group of pixels from the input, in embodiments where the input is an image. A set of neurons of a CNN could receive respective inputs that are determined by applying the same filter to an input. Additionally or alternatively, a set of neurons of a CNN could be associated with respective different filters and could receive respective inputs that are determined by applying the respective filter to the input. Such filters could be trained during training of the CNN or could be pre-specified. For example, such filters could represent wavelet filters, center-surround filters, biologically-inspired filter kernels (e.g., from studies of animal visual processing receptive fields), or some other pre-specified filter patterns.

A CNN or other variety of ANN could include multiple convolutional layers (e.g., corresponding to respective different filters and/or features), pooling layers, rectification layers, fully connected layers, or other types of layers. Convolutional layers of a CNN represent convolution of an input image, or of some other input (e.g., of a filtered, downsampled, or otherwise-processed version of an input image), with a filter. Pooling layers of a CNN apply non-linear downsampling to higher layers of the CNN, e.g., by applying a maximum, average, L2-norm, or other pooling function to a subset of neurons, outputs, or other features of the higher layer(s) of the CNN. Rectification layers of a CNN apply a rectifying nonlinear function (e.g., a non-saturating activation function, a sigmoid function) to outputs of a higher layer. Fully connected layers of a CNN receive inputs from many or all of the neurons in one or more higher layers of the CNN. The outputs of neurons of one or more fully connected layers (e.g., a final layer of an ANN or CNN) could be used to determine information about areas of an input image (e.g., for each of the pixels of an input image) or for the image as a whole.

Neurons in a CNN can be organized according to corresponding dimensions of the input. For example, where the input is a sequence of token (a one-dimensional input, with each token representing one or more words, or fractions of words, in an input text string), neurons of the CNN (e.g., of an input layer of the CNN, of a pooling layer of the CNN) could correspond to locations in the one-dimensional input string/sequence. Connections between neurons and/or filters in different layers of the CNN could be related to such locations.

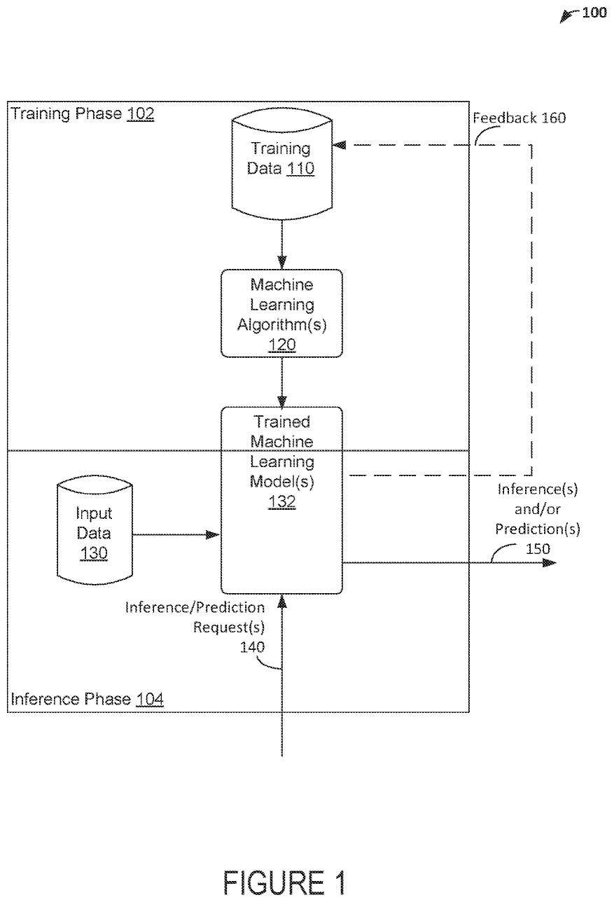

FIG. 1 shows diagram 100 illustrating a training phase 102 and an inference phase 104 of trained machine learning model(s) 132, in accordance with example embodiments. Some machine learning techniques involve training one or more machine learning algorithms, on an input set of training data to recognize patterns in the training data and provide output inferences and/or predictions about (patterns in the) training data. Such output could take the form of filtered or otherwise modified versions of the input, e.g., an input sequence that represents text in a source language could be modified by the machine learning model into (i) an output sequence that represents text in a target language that has similar meaning or semantic content as the input sequence and/or (ii) an output set of embedding vectors that represent, in a semantic embedding space, the meaning or semantic content of the input sequence. The resulting trained machine learning algorithm can be termed as a trained machine learning model. For example, FIG. 1 shows training phase 102 where one or more machine learning algorithms 120 are being trained on training data 110 to become trained machine learning model 132. Then, during inference phase 104, trained machine learning model 132 can receive input data 130 and one or more inference/prediction requests 140 (perhaps as part of input data 130) and responsively provide as an output one or more inferences and/or predictions 150.

As such, trained machine learning model(s) 132 can include one or more models of one or more machine learning algorithms 120. Machine learning algorithm(s) 120 may include, but are not limited to: an artificial neural network (e.g., a herein-described convolutional neural networks, a recurrent neural network, a Bayesian network, a hidden Markov model, a Markov decision process, a logistic regression function, a support vector machine, a suitable statistical machine learning algorithm, and/or a heuristic machine learning system), a support vector machine, a regression tree, an ensemble of regression trees (also referred to as a regression forest), a decision tree, an ensemble of decision trees (also referred to as a decision forest), or some other machine learning model architecture or combination of architectures. For example, the trained machine learning model(s) 132 could include a plurality of artificial neural networks and other elements related to such networks (e.g., mixing or weighting matrices, sums, products, feedforward connections) arranged according to the multi-layer and sublayer architecture of a Transformer or similar model architecture designed to process input sequences. Machine learning algorithm(s) 120 may be supervised or unsupervised, and may implement any suitable combination of online and offline learning.

In some examples, machine learning algorithm(s) 120 and/or trained machine learning model(s) 132 can be accelerated using on-device coprocessors, such as graphic processing units (GPUs), tensor processing units (TPUs), digital signal processors (DSPs), and/or application specific integrated circuits (ASICs). Such on-device coprocessors can be used to speed up machine learning algorithm(s) 120 and/or trained machine learning model(s) 132. In some examples, trained machine learning model(s) 132 can be trained, reside and execute to provide inferences on a particular computing device, and/or otherwise can make inferences for the particular computing device.

During training phase 102, machine learning algorithm(s) 120 can be trained by providing at least training data 110 as training input using unsupervised, supervised, semi-supervised, and/or reinforcement learning techniques. Unsupervised learning involves providing a portion (or all) of training data 110 to machine learning algorithm(s) 120 and machine learning algorithm(s) 120 determining one or more output inferences based on the provided portion (or all) of training data 110. Supervised learning involves providing a portion of training data 110 to machine learning algorithm(s) 120, with machine learning algorithm(s) 120 determining one or more output inferences based on the provided portion of training data 110, and the output inference(s) are either accepted or corrected based on correct results associated with training data 110. In some examples, supervised learning of machine learning algorithm(s) 120 can be governed by a set of rules and/or a set of labels for the training input, and the set of rules and/or set of labels may be used to correct inferences of machine learning algorithm(s) 120.

Semi-supervised learning involves having correct results for part, but not all, of training data 110. During semi-supervised learning, supervised learning is used for a portion of training data 110 having correct results, and unsupervised learning is used for a portion of training data 110 not having correct results. Reinforcement learning involves machine learning algorithm(s) 120 receiving a reward signal regarding a prior inference, where the reward signal can be a numerical value. During reinforcement learning, machine learning algorithm(s) 120 can output an inference and receive a reward signal in response, where machine learning algorithm(s) 120 are configured to try to maximize the numerical value of the reward signal. In some examples, reinforcement learning also utilizes a value function that provides a numerical value representing an expected total of the numerical values provided by the reward signal over time. In some examples, machine learning algorithm(s) 120 and/or trained machine learning model(s) 132 can be trained using other machine learning techniques, including but not limited to, incremental learning and curriculum learning.

In some examples, machine learning algorithm(s) 120 and/or trained machine learning model(s) 132 can use transfer learning techniques. For example, transfer learning techniques can involve trained machine learning model(s) 132 being pre-trained on one set of data and additionally trained using training data 110. More particularly, machine learning algorithm(s) 120 can be pre-trained on data from one or more computing devices and a resulting trained machine learning model provided to computing device CD1, where CD1 is intended to execute the trained machine learning model during inference phase 104. Then, during training phase 102, the pre-trained machine learning model can be additionally trained using training data 110, where training data 110 can be derived from kernel and non-kernel data of computing device CD1. This further training of the machine learning algorithm(s) 120 and/or the pre-trained machine learning model using training data 110 of CDU's data can be performed using either supervised or unsupervised learning. Once machine learning algorithm(s) 120 and/or the pre-trained machine learning model has been trained on at least training data 110, training phase 102 can be completed. The trained resulting machine learning model can be utilized as at least one of trained machine learning model(s) 132.

In particular, once training phase 102 has been completed, trained machine learning model(s) 132 can be provided to a computing device, if not already on the computing device. Inference phase 104 can begin after trained machine learning model(s) 132 are provided to computing device CD1.

During inference phase 104, trained machine learning model(s) 132 can receive input data 130 and generate and output one or more corresponding inferences and/or predictions 150 about input data 130. As such, input data 130 can be used as an input to trained machine learning model(s) 132 for providing corresponding inference(s) and/or prediction(s) 150 to kernel components and non-kernel components. For example, trained machine learning model(s) 132 can generate inference(s) and/or prediction(s) 150 in response to one or more inference/prediction requests 140. In some examples, trained machine learning model(s) 132 can be executed by a portion of other software. For example, trained machine learning model(s) 132 can be executed by an inference or prediction daemon to be readily available to provide inferences and/or predictions upon request. Input data 130 can include data from computing device CD1 executing trained machine learning model(s) 132 and/or input data from one or more computing devices other than CD1.

Input data 130 can include a collection of text strings provided by one or more sources. The collection of text strings can include natural language, artificially generated language, text from books, texts from online forums or chats, texts from emails, and/or other text. Other types of input data are possible as well.

Inference(s) and/or prediction(s) 150 can include output text strings, output token sequences, output sets of embedding vectors, numerical values, and/or other output data produced by trained machine learning model(s) 132 operating on input data 130 (and training data 110). In some examples, trained machine learning model(s) 132 can use output inference(s) and/or prediction(s) 150 as input feedback 160. Trained machine learning model(s) 132 can also rely on past inferences as inputs for generating new inferences.

### III. Illustrative Systems

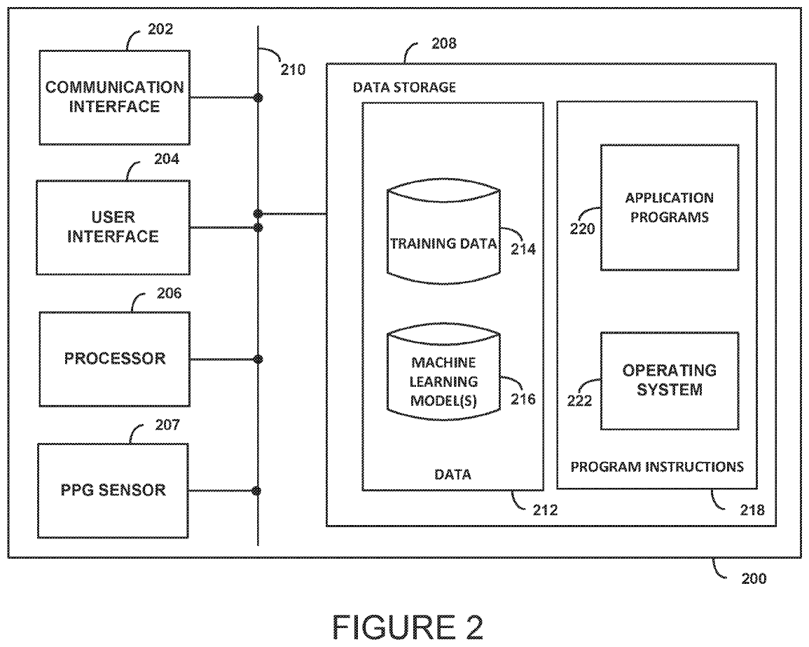

FIG. 2 illustrates an example computing system 200 that may be used to implement the methods described herein. By way of example and without limitation, computing system 200 may be a cellular mobile telephone (e.g., a smartphone), a computer (such as a desktop, notebook, tablet, or handheld computer, a server), elements of a cloud computing system, a robot, a drone, an autonomous vehicle, or some other type of device. It should be understood that computing system 200 may represent a physical computing device such as a server, a particular physical hardware platform on which a machine learning application operates in software, or other combinations of hardware and software that are configured to carry out machine learning or other functions as described herein. The computing system 200 could be a central system (e.g., a server, elements of a cloud computing system) that is configured to receive PPG signals, demographic and/or medical information, or other information from a remote system (e.g., a computing system in a physician's or radiologist's office or clinic, from a watch or other wearable device, from a user's phone) and to responsively transmit, to that remote system or to some other system, output feature sets, heart rate values, cardiovascular disease risk predictions or other diagnoses, or other information generated by applying the PPG signal(s) and/or associated information to a machine learning model or other predictive algorithm as described herein. Additionally or alternatively, the computing system 200 could be such a remote system, configured to transmit PPG signals or other information to a central system, receive output feature sets, diagnostic information, or other information in response, and/or to take some other actions as described herein.

As shown in FIG. 2, computing system 200 may include a communication interface 202, a user interface 204, a processor 206, a PPG sensor 207, and data storage 208, all of which may be communicatively linked together by a system bus, network, or other connection mechanism 210.

Communication interface 202 may function to allow computing system 200 to communicate, using analog or digital modulation of electric magnetic, electromagnetic, optical, or other signals, with other devices, access networks, and/or transport networks. Thus, communication interface 202 may facilitate circuit-switched and/or packet-switched communication, such as plain old telephone service (POTS) communication and/or Internet protocol (IP) or other packetized communication. For instance, communication interface 202 may include a chipset and antenna arranged for wireless communication with a radio access network or an access point. Also, communication interface 202 may take the form of or include a wireline interface, such as an Ethernet, Universal Serial Bus (USB), or High-Definition Multimedia Interface (HDMI) port. Communication interface 202 may also take the form of or include a wireless interface, such as a Wifi, BLUETOOTH®, global positioning system (GPS), or wide-area wireless interface (e.g., WiMAX or 3GPP Long-Term Evolution (LTE)). However, other forms of physical layer interfaces and other types of standard or proprietary communication protocols may be used over communication interface 202. Furthermore, communication interface 202 may comprise multiple physical communication interfaces (e.g., a Wifi interface, a BLUETOOTH® interface, and a wide-area wireless interface).

In some embodiments, communication interface 202 may function to allow computing system 200 to communicate with other devices, remote servers, access networks, and/or transport networks.

User interface 204 may function to allow computing system 200 to interact with a user or other entity, for example to receive input from and/or to provide output to the user. Thus, user interface 204 may include input components such as a keypad, keyboard, touch-sensitive or presence-sensitive panel, computer mouse, trackball, joystick, microphone, and so on. User interface 204 may also include one or more output components such as a display screen which, for example, may be combined with a presence-sensitive panel. The display screen may be based on CRT, LCD, and/or LED technologies, or other technologies now known or later developed. User interface 204 may also be configured to generate audible output(s), via a speaker, speaker jack, audio output port, audio output device, earphones, and/or other similar devices.

Processor 206 may comprise one or more general purpose processors—e.g., microprocessors—and/or one or more special purpose processors—e.g., digital signal processors (DSPs), graphics processing units (GPUs), floating point units (FPUs), network processors, tensor processing units (TPUs), or application-specific integrated circuits (ASICs). In some instances, special purpose processors may be capable of operating the PPG sensor 207 to generate PPG signals, executing machine learning models, training machine learning models, among other applications or functions. Data storage 208 may include one or more volatile and/or non-volatile storage components, such as magnetic, optical, flash, or organic storage, and may be integrated in whole or in part with processor 206. Data storage 208 may include removable and/or non-removable components.

Processor 206 may be capable of executing program instructions 218 (e.g., compiled or non-compiled program logic and/or machine code) stored in data storage 208 to carry out the various functions described herein. Therefore, data storage 208 may include a non-transitory computer-readable medium, having stored thereon program instructions that, upon execution by computing system 200, cause computing system 200 to carry out any of the methods, processes, or functions disclosed in this specification and/or the accompanying drawings. The execution of program instructions 218 by processor 206 may result in processor 206 using data 212.

By way of example, program instructions 218 may include an operating system 222 (e.g., an operating system kernel, device driver(s), and/or other modules) and one or more application programs 220 (e.g., functions for executing and/or training a machine learning model) installed on computing system 200. Data 212 may include training data (e.g. medical diagnostic images and associated labels, medical records, etc.) 214 and/or machine learning model(s) 216 that may be determined therefrom or obtained in some other manner.

Application programs 220 may communicate with operating system 222 through one or more application programming interfaces (APIs). These APIs may facilitate, for instance, application programs 220 transmitting or receiving information via communication interface 202, receiving and/or displaying information on user interface 204, and so on.

Application programs 220 may take the form of “apps” that could be downloadable to computing system 200 through one or more online application stores or application markets (via, e.g., the communication interface 202). However, application programs can also be installed on computing system 200 in other ways, such as via a web browser or through a physical interface (e.g., a USB port) of the computing system 200.

### IV. Example Methods

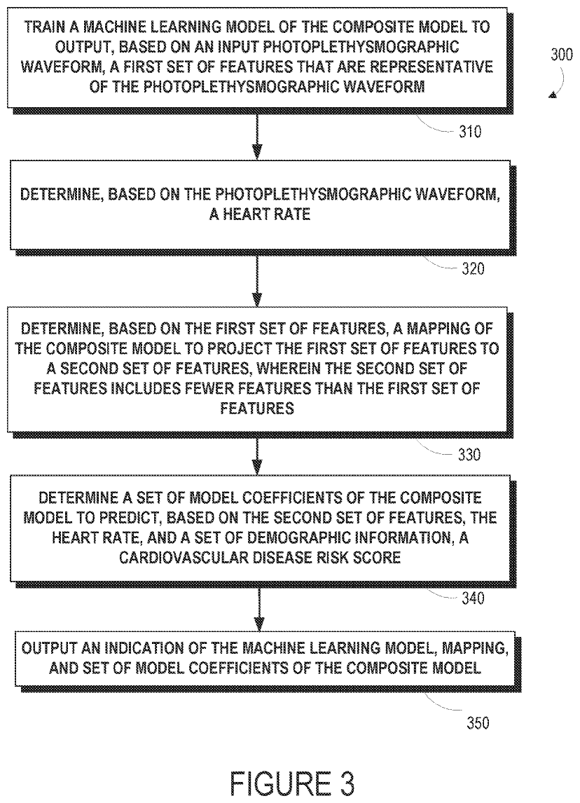

FIG. 3 is a flowchart of an example computer-implemented method 300. The method 300 includes training a machine learning model of the composite model to output, based on an input photoplethysmographic waveform, a first set of features that are representative of the photoplethysmographic waveform (310). The method 300 additionally includes determining, based on the photoplethysmographic waveform, a heart rate (320). The method 300 additionally includes determining, based on the first set of features, a mapping of the composite model to project the first set of features to a second set of features, wherein the second set of features includes fewer features than the first set of features (330). The method 300 additionally includes determining a set of model coefficients of the composite model to predict, based on the second set of features, the heart rate, and a set of demographic information, a cardiovascular disease risk score (340). The method 300 also includes outputting an indication of the machine learning model, mapping, and set of model coefficients of the composite model (350). The method 300 could include additional or alternative features.

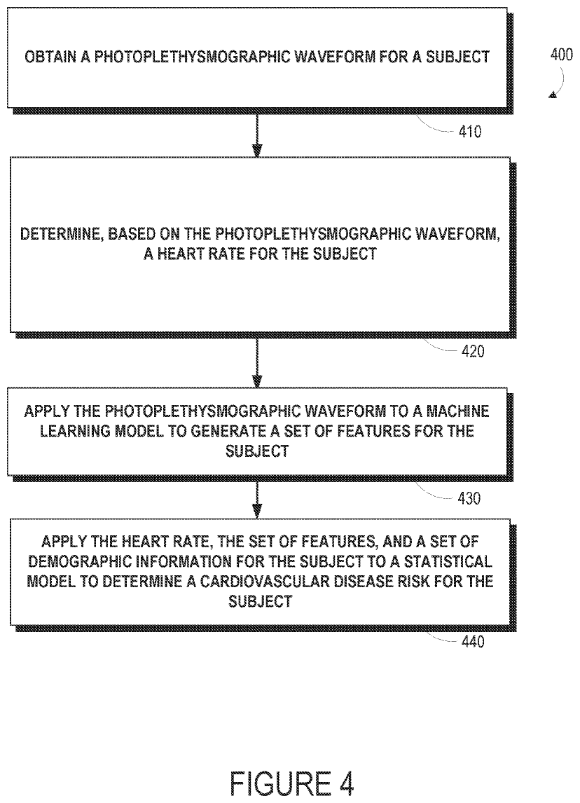

FIG. 4 is a flowchart of an example computer-implemented method 400. The method 400 includes obtaining a photoplethysmographic waveform for a subject (410). The method 400 additionally includes determining, based on the photoplethysmographic waveform, a heart rate for the subject (420). The method 400 additionally includes applying the photoplethysmographic waveform to a machine learning model to generate a set of features for the subject (430). The method 400 additionally includes applying the heart rate, the set of features, and a set of demographic information for the subject to a statistical model to determine a cardiovascular disease risk for the subject (440). The method 400 could include additional or alternative features.

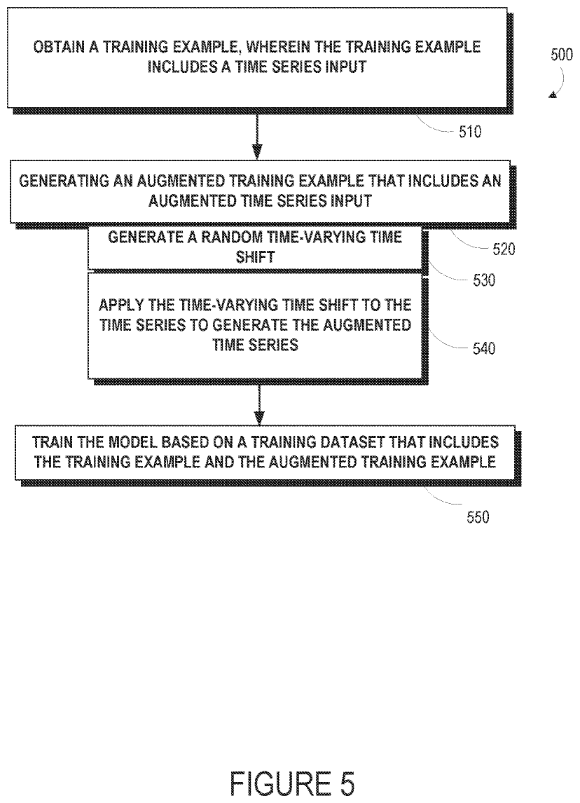

FIG. 5 is a flowchart of an example computer-implemented method 500. The method 500 includes obtaining a training example, wherein the training example includes a time series input (510). The method 300 additionally includes generating an augmented training example that includes an augmented time series input (520). This includes generating a random time-varying time shift (530) and applying the time-varying time shift to the time series to generate the augmented time series (540). The method 500 additionally includes training the model based on a training dataset that includes the training example and the augmented training example (530). The method 500 could include additional or alternative features.

### V. Example Embodiments and Experimental Results

The embodiments described herein were developed into a number of example embodiments, which are described in greater detail in this section. Some of these example embodiments were experimentally evaluated, and the results of such experimentation is also provided in this section.

Cardiovascular diseases (CVDs) are responsible for one third of deaths globally with approximately three quarters occurring in low- and middle-income countries (LMICs) where there is a paucity of resources for early disease detection. Because CVD risk factors such as hypertension, diabetes, or hyperlipidemia are typically symptomless before advanced disease, there is a need for screening programs to identify those at high risk of CVD events. Interventions such as lifestyle counseling, with or without prescription medications, have shown to be an effective strategy for CVD prevention among these individuals.

Multiple risk scores, such as the WHO/ISH risk chart and Globorisk scores, have been developed to triage CVD risk based on demographics, past medical history, vital signs, and laboratory data. However, the dependency of these risk scores on medical and laboratory equipment (e.g., sphygmomanometers) limits their reach. Specifically, low-resource healthcare systems rely largely on opportunistic screening, such as via community healthcare workers (CHWs), to close access gaps. The low-cost, easy-to-use, lightweight, digital point-of-care tools described herein, which use PPG sensors that are commonly available (e.g., that are widely available in smartphones, can further the reach and capability of CHW-based programs and facilitate large-scale screening at low cost.

Among sensing signals for the circulatory system, photoplethysmography (PPG) is a non-invasive, fast, simple, and low-cost technology, and can be captured with sensors available on increasingly ubiquitous devices such as smartphones and pulse oximeters. PPG measures the change in blood volume in an area of tissue across cardiac cycles and can be used for heart rate monitoring in healthcare settings. PPG can be measured from smartphone cameras by placing a finger on the camera. CVD-risk estimation based on PPG signals as described herein can provide an accurate, highly accessible screening tool in low-resource health systems.

The efficacy of leveraging PPG for CVD risk prediction as described herein was investigated using data from the UK Biobank (UKB). Specifically, ten-year risk of developing a major adverse cardiovascular event (MACE) was predicted using deep learning-based PPG embeddings and heart rate (measured by PPG) along with other demographics, including age, sex and smoking status, but without any inputs from physical examination or laboratory data. The deep learning PPG-based CVD risk prediction score (which may be referred to as “DLS” herein) generated as described herein was well-calibrated and non-inferior to the existing comparative office-based CVD risk score using predictors from WHO/ISH and Globorisk, such office-based scores requiring blood pressure, weight and height measurement, an/dor laboratory data.

### Methods

The new CVD risk prediction score, DLS, was developed using age, sex, smoking status, and the results of analysis of PPG signals using deep learning models as described herein. A Cox proportional hazard model and data from the UKB were used to predict the ten-year risk of MACE among individuals free of CVD at baseline.

The DLS was developed and evaluated using data from the UKB dataset, filtered to focus on participants aged 40-74. UKB participants who had PPG waveforms recorded were stratified into three subsets: train (n=105,319), tune (n=46,868), and test (n=57,702) subsets based on geographic information on the site of data collection, i.e., latitude and longitude. This strategy comports with TRIPOD guidelines on external validation (specifically validation on a different geographic region) by allowing for non-random variation between data splits such as differences in data acquisition or environment.

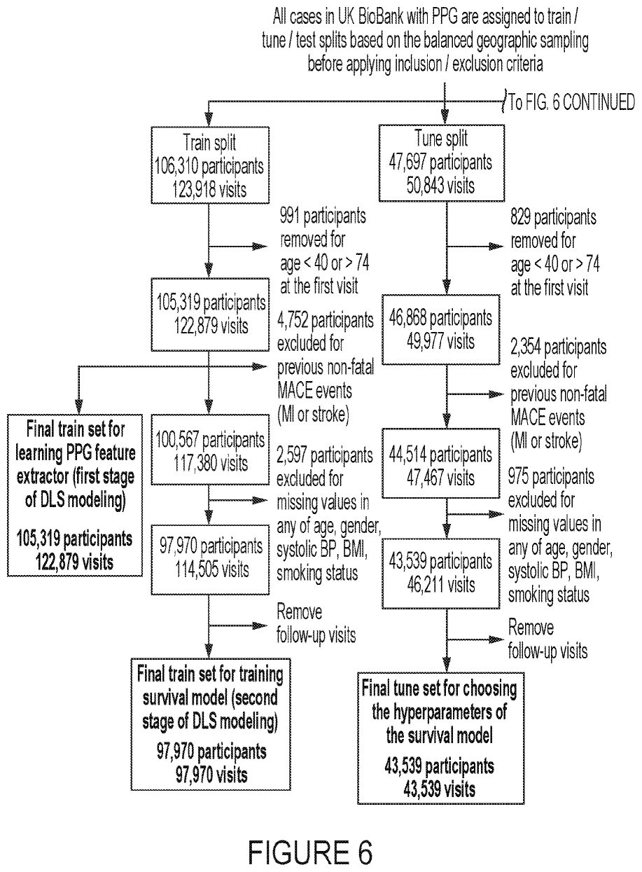

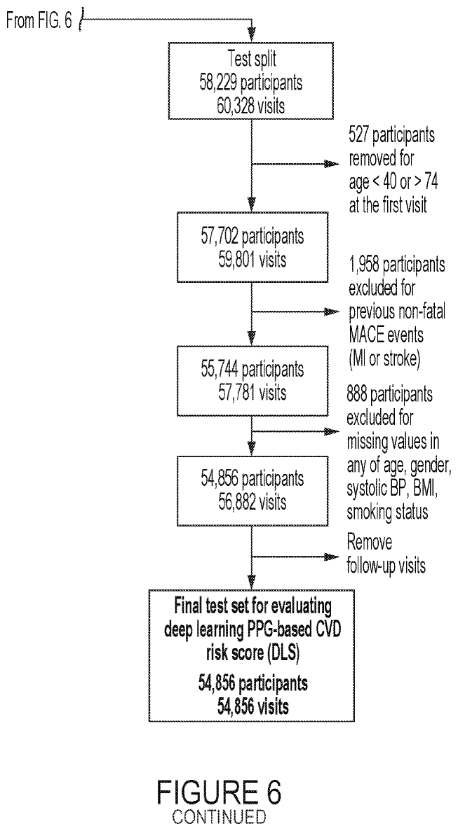

PPG waveforms from all visits for the participants were used in the training subset to train the PPG feature extractor in DLS. The low-dimensional numeric outputs (embeddings) computed by this model were used as additional input features to the Cox model. To develop the Cox model that generates DLS to predict MACE risk, additional clinical and demographic variables and inclusion/exclusion criteria were added. Participants with non-fatal myocardial infarction or stroke before their first visit, or that were missing any of the variables for the model (age, sex, and smoking status), were excluded. Those without body mass index (BMI) or systolic BP (SBP) were also excluded for a fair comparison against the other office- and lab-based risk prediction models. For each participant, only the measurements related to their first visit were included. All numerically measured variables were standard-scaled. Cox models were regularized using a ridge penalty. In the final cohort, 97,970, 43,539, and 54,856 participants were included to train, tune, and test the survival model, respectively (FIG. 6). The descriptive statistics of this cohort are listed in Table 1.

TABLE 1
Cohort statistics for 10-year major adverse cardiovascular events (MACE) risk prediction at the first UK Biobank visit.

| | Overall | | Train | | Tune | | Test | |
|---|---|---|---|---|---|---|---|---|
| Number of sites | 15 | | 9 | | 3 | | 3 | |
| Geographic location | See train/tune/test | | Swansea, Bristol, | | Newcastle, | | Croydon, Hounslow, | |
| of sites | | | Birmingham, | | Middlesborough, | | Reading | |
| | | | Nottingham, | | Liverpool | | | |
| | | | Sheffield, Cheadle, | | | | | |
| | | | Wrexham | | | | | |
| Patients | 196,365 | | 97,970 | | 43,539 | | 54,856 | |
| MACE, count (%) | 5,650 | (2.9) | 2,798 | (2.9) | 1,401 | (3.2) | 1,451 | (2.6) |
| Age, median [IQR] | 59.0 | [51.0, 64.0] | 59.0 | [52.0, 65.0] | 60.0 | [52.0, 64.0] | 58.0 | [50.0, 63.0] |
| Sex = female, | 107,679 | (54.8) | 52,885 | (54.0) | 23,833 | (54.7) | 30,961 | (56.4) |
| count (%) | | | | | | | | |
| Smoking status, | 84,111 | (42.8) | 41,559 | (42.4) | 18,436 | (42.3) | 24,116 | (44.0) |
| count (%) | | | | | | | | |
| BMI, median [IQR] | 26.6 | [24.0, 29.7] | 26.6 | [24.1, 29.7] | 26.9 | [24.3, 30.0] | 26.2 | [23.7, 29.4] |
| SBP, median [IQR] | 136.5 | [124.5, 149.5] | 137.0 | [125.5, 150.0] | 139.0 | [127.0, 152.0] | 133.5 | [122.0, 146.5] |
| Total cholesterol, | 5.7 | [5.0, 6.5] | 5.7 | [5.0, 6.5] | 5.7 | [5.0, 6.5] | 5.6 | [4.9, 6.4] |
| median [IQR] | | | | | | | | |
| HDL, median [IQR] | 1.4 | [1.2, 1.7] | 1.4 | [1.2, 1.7] | 1.4 | [1.2, 1.7] | 1.4 | [1.2, 1.7] |
| HbA1c, median [IQR] | 35.3 | [32.9, 38.0] | 35.3 | [32.9, 37.9] | 35.3 | [32.9, 37.9] | 35.4 | [32.9, 38.2] |
| Diabetes, count (%) | 4738 | (2.4) | 2129 | (2.2) | 1063 | (2.4) | 1546 | (2.8) |
| Hypertension, | 52,299 | (26.6) | 26,097 | (26.6) | 11,618 | (26.7) | 14,584 | (26.6) |
| count (%) | | | | | | | | |
| History of angina, | 161 | (0.1) | 89 | (0.1) | 34 | (0.1) | 38 | (0.1) |
| count (%) | | | | | | | | |
| History of | 18,872 | (9.6) | 8,517 | (8.7) | 4,380 | (10.1) | 5,975 | (10.9) |
| hyperlipidemia, | | | | | | | | |
| count (%) | | | | | | | | |
| On hypertension | 39762 | (20.2) | 20072 | (20.5) | 9151 | (21.0) | 10539 | (19.2) |
| medication, median | | | | | | | | |
| [IQR] | | | | | | | | |

| | | | | | | | |
|---|---|---|---|---|---|---|---|
| On statin, median | 32297 | (16.4) | 16068 | (16.4) | 7431 | (17.1) | MMMMMM [symbol] |
| [IQR] | | | | | | | 0 |
| | | | | | | | [symbol] |

[symbol]

indicates data missing or illegible when filed

### First Stage Model Development: PPG Feature Extractor

For DLS, a deep learning-based feature extractor was trained to learn PPG representations from raw PPG waveform signals, using a one-dimensional ResNet18 as the neural network architecture. The feature extractor was trained on the train subset, network weights were picked that maximized the Cox pseudolikelihood on the tune subset. These weights were used to compute PPG embeddings on the train, tune, and test subsets. The PPG embeddings were further processed by principal component analysis (PCA) to five PCA-derived DLS features that were used by the survival model.

### Second State Model Development: Survival Model

In the second stage, a Cox proportional hazards regression model was developed to predicting ten-year MACE risk, using as inputs age, sex, smoking status, PCA-derived PPG embeddings, and PPG-HR (heart rate measured during PPG assessment). The model was trained on the train subset and tuned on the tune subset to decide the best-performing ridge regularization parameter.

### Models for Comparison/Reference

For comparisons, different survival models were developed based on different feature sets (Table 2 “Features used” column), including office-based and laboratory-based refit-WHO scores using the CVD risk predictors adopted in WHO/ISH risk chart and Globorisk studies, metadata-only model (age, sex, smoking status), metadata+PPG morphology (a model with metadata and engineered PPG features describing waveform morphology, such as dicrotic notch presence), a model without smoking status as an input (metadata without smoking, DLS without smoking), and the “Full” model that considered metadata, laboratory data, medication and medical history as a reference, to compute CVD risk score. The model using shared predictors was chosen from the office-based WHO/ISH risk chart and Globorisk score (office-based refit-WHO score) as the main reference since they were adopted in the CVD risk research for low-resource settings. To ensure the fairest comparison the coefficients for the WHO and Globorisk predictors were re-fitted using the same UKB train subset as the DLS, and a sensitivity analysis was conducted using the original coefficients with recalibration.

TABLE 2
Model performance comparison of 10-year major adverse cardiovascular events (MACE) risk prediction between DLS versus other
methods for non-operating point dependent metrics. The primary analysis was non-inferiority of the C-statistic of the DLS
model compared with the office-based refit-WHO model. 95% confidence intervals (CIs) of C-statistic, cfNRI, and slope were
obtained via bootstrapping, and the p-values were computed via a permutation test. The slope was not calculated for SBP-
140 since its output is binary. In the “Features used” column, “Metadata” includes age, sex, and smoking status.

| | | | P-value | P-value | | | | | |
|---|---|---|---|---|---|---|---|---|---|
| | | | for non- | for | | | cfNRI | | |
| | C- | | inferiority | superiority | | cfNRI | (non- | | |
| | statistic | | of C- | of C- | cfNRI | (event) | event) | Calibration | Features |
| Model | (%) | Delta (%) | statistic | statistic | (%) | (%) | (%) | slope | used* |
| Office-based | 70.9 | n/a (reference) | | | | | | 0.979 | Metadata + |
| refit-WHO | (69.7, 72.2) | | | | | | | (0.915, 1.038) | BMI + SBP |
| DLS | 71.1 | 0.2 | <0.01 | 0.292 | 0.1 | 0.1 | 0.0 | 0.981 | Metadata + |
| | (69.9, 72.4) | (−0.4, 0.8) | | | (0.0, 0.1) | (0.0, 0.2) | (0.0, 0.0) | (0.919, 1.045) | PPG |
| Metadata | 69.1 | −1.7 | <0.01 | 1 | −0.4 | −0.2 | 0.2 | 0.94 | Metadata |
| | (67.9, 70.4) | (−2.2, −1.3) | | | (−0.5, −0.3) | (−0.3, −0.2) | (0.1, 0.2) | (0.875, 1.004) | |
| SBP-140 | 59.4 | −11.5 | 1 | 1 | −1.3 | −1.0 | 0.3 | — | SBP |
| | (58.3, 60.5) | (−12.7, −10.2) | | | (−1.4, −1.2) | (−1.1, −0.9) | (0.3, 0.3) | | |

Evaluated on the subset with all PPG morphology data available

| Metadata + | 70.0 | −0.9 | <0.01 | 1 | −0.1 | −0.1 | 0.1 | 1.02 | Metadata + |
|---|---|---|---|---|---|---|---|---|---|
| PPG | (68.8, 71.3) | (−1.4, −0.4) | | | (−0.2, −0.1) | (−0.1, 0.0) | (0.1, 0.1) | (0.951, 1.086) | engineered |
| morphology | | | | | | | | | PPG features |
| Office-based | 70.9 | n/a (subset reference) | | | | | | 0.977 | (Metadata + |
| refit-WHO | (69.7, 72.2) | | | | | | | (0.913, 1.035) | BMI + SBP) |

Evaluated on the subset with laboratory data available

| Lab-based | 71.6 | 0.5 | <0.01 | <0.01 | 0.2 | 0.3 | 0.1 | 0.921 | Metadata + |
|---|---|---|---|---|---|---|---|---|---|
| refit-WHO | (70.4, 72.9) | (0.1, 0.9) | | | (0.1, 0.2) | (0.2, 0.3) | (0.1, 0.1) | (0.864, 0.982) | total |
| | | | | | | | | | cholesterol + |
| | | | | | | | | | glucose |
| Office-based | 71.1 | n/a (subset reference) | | | | | | 0.897 | (Metadata + |
| refit-WHO | (69.9, 72.4) | | | | | | | (0.838, 0.959) | BMI + SBP) |

DLS was further developed into DLS+(DLS with BMI), and DLS++(DLS with BMI and SBP) that included additional non-laboratory, office-based measurements as inputs of the survival model to better investigate the prognostic value of PPG on top of the existing office-based refit-WHO model.

All models were trained on the same train subset and tuned on the tune subset except for laboratory-based refit-WHO score, metadata+PPG morphology, and the Full models that were trained, tuned, and compared based on a subset of the testing data without missing values of the input features.

### Evaluation

The outcome, ten-year risk of MACE, was defined as a composite outcome of three components, non-fatal myocardial infarction, stroke, and CVD-related death (using ICD codes and cause of death to identify). To define the outcome, (1) the date of heart attack, myocardial infarction, stroke, ischemic stroke, either diagnosed by doctor or self-reported, (2) the record of ICD-10 (international classification of diseases, 10th revision) clinical codes, and (3) strings that are associated with the CVD-related death were used. The ICD-10 codes used included 121 (acute myocardial infarction), 122 (subsequent myocardial infarction), 123 (complications after myocardial infarction), 163 (cerebral infarction), 164 (stroke not specified as hemorrhage or infarction). The strings used for matching include those related to coronary artery diseases, myocardial infarction, stroke, hypertensive diseases, heart failure, thromboembolism, arrhythmia, valvular diseases and other heart problems. The earliest date on any of the data sources mentioned above was used as the outcome date.

For primary analysis, DLS was compared with the office-based refit-WHO score, which is a risk model for healthy individuals across different countries, using Harrell's C-statistic. A non-inferiority test was performed with a pre-specified margin of 2.5% and alpha of 0.05, both selected based on power simulations using the tune subset. For secondary analyses, DLS was also compared with scores generated by other models mentioned above.

Additional evaluation metrics included the category-free net reclassification improvement (cfNRI), and after defining a specific risk threshold (model operating point), sensitivity, specificity, NRI, and adjusted hazard ratio (HRs). For NRI and cfNRI, the respective event and non-event components were also reported. Risk thresholds were selected in three ways: (1) matching the sensitivity of SBP-140 (described next), (2) matching the specificity of SBP-140, and (3) the 10% predicted risk threshold suggested by the Globorisk study. Elevated SBP above 140 mmHg (“SBP-140”) was used for threshold selection because it was used as a simple single-visit indicator of BP control in the healthcare program of some countries such as India. To calculate sensitivity and specificity, the participants without a ten-year follow up were excluded if they didn't have a MACE event within ten years. To evaluate model calibration, the slope of the line was used to compare predicted and actual event rates, for deciles of model prediction. Subgroup analyses were performed based on smoking status, sex, age, elevated HbA1c, and hypertension status. Quintiles were used for the elevated HbA1c subgroup due to the smaller sample size.

For statistical precision, the Clopper-Pearson exact method was used to compute the 95% confidence intervals (CIs) for sensitivity and specificity, and the non-parametric bootstrap method was used with 1,000 iterations to compute 95% of all remaining metrics and delta values. For hypothesis tests in secondary and exploratory analysis, a permutation test was used to examine the non-inferiority and superiority of the C-statistic, and the one-sided Wald test for sensitivity and specificity. The log-rank test was used to determine whether survival differed between the model-defined low and high risk groups. For all two-sided tests, we used an alpha value of 0.05.

### Results

DLS demonstrated non-inferiority to the office-based refit-WHO score. The ten-year MACE risk prediction performance of all methods was evaluated using the UKB test subset, which was held-out during the training process. The DLS yielded a C-statistic of 71.1% (95% CI [69.9, 72.4]). When compared with the office-based refit-WHO score, the DLS was non-inferior (p<0.01), with a delta of +0.2% (−0.4, 0.8). The cfNRI was 0.1% (0.0, 0.1), stemming primarily from improved reclassification of events (0.1% [0.0, 0.2]), without performance penalty in the non-events (0.0% [0.0, 0.0]).

Based on the C-statistic, there was an incremental improvement when the metadata model (69.1%) was augmented with manually engineered (not deep learning derived) PPG morphology features (70.0%). The DLS was superior to this metadata+PPG morphology features model (p<0.01), indicating value in deep learning based feature extraction. The lab-based model (which included as inputs total cholesterol and glucose information) was superior to the office-based refit-WHO score (71.6% versus 70.9%, p<0.01). By applying the Globorisk scores, recalibrated on the UKB cohort for baseline hazard and mean risk factors but without re-estimating the coefficients, the office-based Globorisk yielded a C-statistic of 70.0% (68.8, 71.2), and the lab-based Globorisk yielded a C-statistic of 69.8% (68.5, 71.1). More details are shown in Table 2.

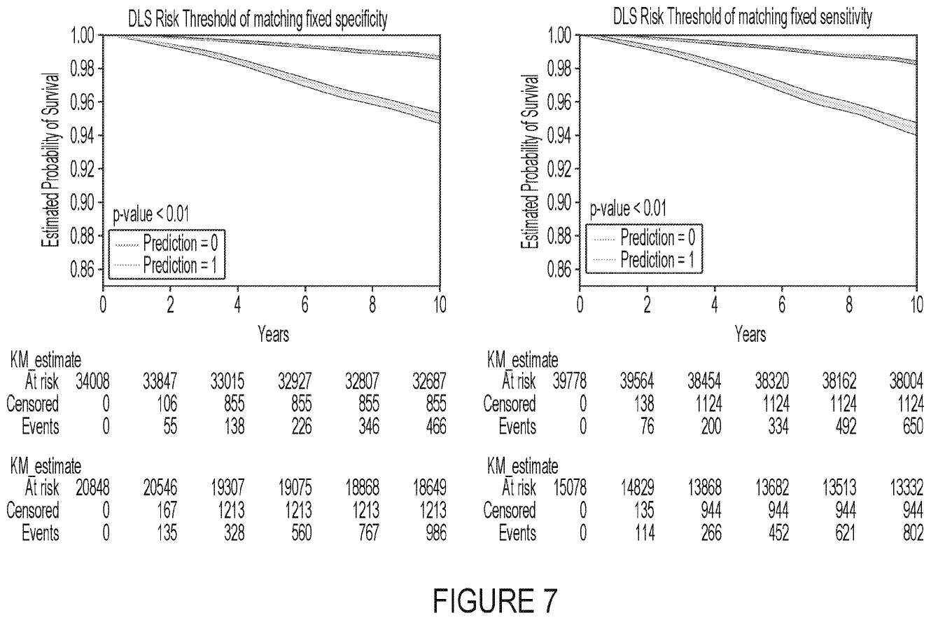

For a fair comparison, risk thresholds were selected that matched the specificity or sensitivity of SBP-140 (specificity of 63.7%, sensitivity of 55.2%). At matched specificity, the sensitivity of the DLS (67.9%) was non-inferior to the office-based refit-WHO score (67.7%) (p=0.012), and a comparable NRI, while the metadata and metadata+PPG morphology models were not (p=0.984 and p=0.305, respectively). At the matched sensitivity, the DLS's specificity (74.0%) was also non-inferior to the baseline (73.1%) (showed superiority with p<0.01), with a comparable NRI. The laboratory-based refit-WHO and the model using metadata and PPG morphology-based features were also non-inferior to the office-based refit-WHO score, despite these models requiring additional inputs from laboratory measurements or engineered PPG features, respectively. The metadata-only model performed more poorly than the office-based refit-WHO score across different metrics. Kaplan Meier analysis was also performed on risk groups defined using the above approach (FIG. 7). Both thresholds showed significant (p<0.01, log rank tests) differences between the groups.

In addition to the default set of inputs to the DLS, models with BMI, and with both BMI and SBP (both of which are predictors in the office-based refit-WHO) included as additional inputs, were also evaluated. These expanded DLS model were referred to as DLS+ and DLS++, respectively. Adding BMI (DLS+) improved DLS in terms of both discrimination and net reclassification. Additional improvement was observed after adding SBP (DLS++), which further improved the DLS model, and demonstrated superiority across different metrics. For DLS and its variants (DLS+ and DLS++), the cfNRI and NRI with different risk thresholds were also on par with the office-based refit-WHO score. These findings indicate that combining the existing non-laboratory risk factors from the refit-WHO score with the DLS features yields a more accurate CV risk estimation. A model (the “Full” model) was also developed that includes more of the risk factors used in CVD risk scores commonly used in high-income countries (QRISK and/or ASCVD), as well as a model that incorporates genetic risk.

The association between each model and MACE via the coefficients and hazard ratios (HRs) was also analyzed. In the office-based refit-WHO score, smoking, older age, higher BMI, and higher SBP were associated with the ten-year MACE risk. Some DLS features were also associated with the ten-year MACE risk (p<0.05 for four deep learning PPG features in DLS and DLS+, and for two PPG features for DLS++).

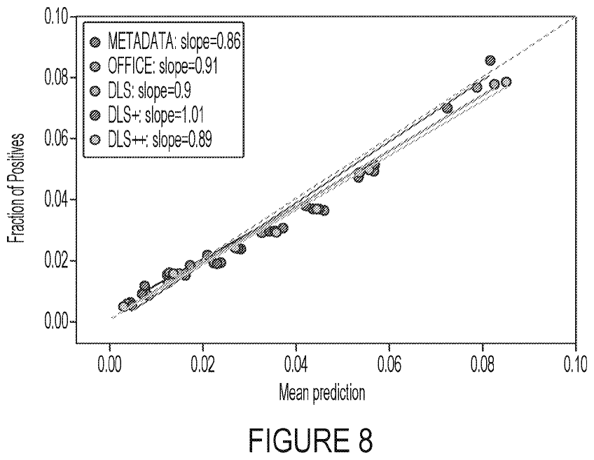

The predicted and observed risks of ten-year MACE were similar across different models (FIG. 8), indicating that DLS has similar calibration performance compared with other models. The calibration slope of DLS was similar to the office-based refit-WHO score (0.981 versus 0.979) (Table 2). DLS++ has a comparable calibration performance. All models except the DLS+ had an observed ten-year MACE risk estimation within 5% mean absolute calibration error (i.e., the slopes were between 0.95-1.05).

Finally, DLS is on par with the office-based refit-WHO score in some subgroups. DLS demonstrated non-inferiority in some subgroups and showed superiority in smoking, hypertensive and male subgroups. Both the office-based refit-WHO score and DLS had a similar trend of performance. Both models have higher sensitivity and lower calibration error but lower specificity on the smoking, older, male, and hypertensive subgroups. The models were well-calibrated for most subgroups, but systematically overestimated absolute risk about 4.0% in the elevated A1c and about 1.0% in hypertensive subgroups. This finding indicates that the developed risk models tended to be better calibrated and to better predict ten-year MACE risk in a population that has higher known CVD risk factors, such as older, male, smoking, higher blood glucose and hypertensive subgroups. Across different age, sex, smoking, and comorbidity (diabetes and hypertension) subgroups, the calibration for all risk scores were similar in predicting ten-year MACE risk in smoking, age greater than 55, male, not elevated A1c populations, with prediction errors within 10% (i.e. the calibration regression slope between 0.9 and 1.1).

A deep learning PPG-based CVD risk score as described herein, DLS, was developed to predict ten-year MACE risk using age, sex, smoking status, heart rate and deep learning-derived PPG features. Without requiring any vital signs or laboratory measurement, DLS demonstrated non-inferior performance compared to the office-based refit-WHO score with coefficients re-estimated on the same cohort. Results were consistent between metrics (C-statistic, NRI, cfNRI, sensitivity, specificity, calibration slope), and across various subgroups. Improved cfNRI and NRI also indicated the capability of DLS to reclassify cases better than the office-based refit-WHO score. Additionally, when available, adding office-based features (BMI, SBP) on top of DLS further improved the model performance.

Such embodiments, which leverage PPG and deep learning, can play important roles in settings where equipment access to healthcare is limited, such as community-based screening programs in LMCs. The DLS described herein demonstrated performance comparable to that of the re-estimated office-based refit-WHO score, without requiring accurate laboratory examination, vital signs measured via additional devices, or BMI. This feature improves accessibility for health systems that have limited resources to collect vitals and labs for CVD risk screening and triage. Such PPG signals could be captured through a smartphone to facilitate large-scale screening and triage in the community at low cost.

TABLE 3
Model performance comparison of 10-year major adverse cardiovascular events (MACE) risk prediction between
DLS versus each of the other methods using a risk threshold matches the same specificity or sensitivity
of SBP-140. 95% confidence intervals (CIs) of sensitivity and specificity were obtained from the Clopper-
Pearson exact method, and the p-values were calculated by the permutation test with the prespecified
margin of 2.5% and alpha of 0.05. The 95% CIs of NRI were computed via bootstrapping.

| | Sensitivity@specificity of 63.7% | | | | | | |
|---|---|---|---|---|---|---|---|
| | | | Non- | | | | NRI |
| | Mean | Delta | inferiority | Superiority | | NRI | (non-event) |
| Model | (%) | (%) | p-value | p-value | NRI (%) | (event) (%) | (%) |
| Office-based | 67.7 | reference | | | | | |
| refit-WHO | (65.2, 70.1) | | | | | | |
| DLS | 67.9 | 0.1 | <0.01 | 0.654 | −0.3 | 1.0 | 1.2 |
| | (65.4, 70.3) | (−1.9, 2.0) | | | (−2.0, 1.6) | (−0.9, 2.9) | (0.9, 1.5) |
| Metadata | 63.6 | −4.1 | 0.984 | 1 | −2.7 | 0.8 | 3.5 |
| | (61.0, 66.0) | (−5.9, −2.2) | | | (−4.5, −0.9) | (−0.9, 2.6) | (2.9, 3.8) |
| SBP-140 | 55.4 | −12.4 | | | −11.1 | −18.7 | −7.7 |
| | (53.1, 57.9) | (−15.1, −9.3) | 1 | 1 | (−13.6, −8.2) | (−21.2, −15.8) | (−8.1, −7.2) |

Evaluated on the subset with all PPG morphology data available

| Metadata + | 66.0 | −1.8 | 0.305 | 0.992 | −1.6 | 0.3 | 1.9 |
|---|---|---|---|---|---|---|---|
| PPG | (63.5, 68.5) | (−3.5, −0.1) | | | (−3.4, 0.2) | (−1.5, 2.2) | (1.7, 2.2) |
| morphology | | | | | | | |
| Office-based | 67.7 | n/a (subset reference) | | | | | |
| refit-WHO | (65.3, 70.1) | | | | | | |

Evaluated on the subset with laboratory data available

| Lab-based | 69.1 | 1.0 | <0.01 | 0.106 | 2.0 | 4.2 | 2.2 |
|---|---|---|---|---|---|---|---|
| refit-WHO | (66.6, 71.6) | (−0.7, 2.5) | | | (0.3, 3.7) | (2.5, 5.8) | (1.9, 2.4) |
| Office-based | 68.2 | n/a (subset reference) | | | | | |
| refit-WHO | (65.7, 70.7) | | | | | | |

| | Specificity@sensitivity of 55.2% | | | | | | |
|---|---|---|---|---|---|---|---|
| | | | Non- | | | | NRI |
| | Mean | Delta | inferiority | Superiority | | NRI | (non-event) |
| Model | (%) | (%) | p-value | p-value | NRI (%) | (event) (%) | (%) |
| Office-based | 73.1 | reference | | | | | |
| refit-WHO | (72.7, 73.5) | | | | | | |
| DLS | 74.0 | 0.9 | <0.01 | <0.01 | 1.1 | 1.6 | 0.4 |
| | (73.6, 74.4) | (−0.7, 2.5) | | | (−0.9, 3.1) | (−0.5, 3.4) | (0.1, 0.8) |
| Metadata | 70.4 | −2.4 | 0.961 | 1 | −2.4 | −0.4 | 2.0 |
| | (70.0, 70.8) | (−3.9, −1.1) | | | (−4.9, −0.3) | (−3.1, 1.6) | (0.6, 2.7) |
| SBP-140 | 63.6 | −11.0 | | | −6.8 | 12.6 | 16.6 |
| | (63.2, 64.0) | (−74.4, −8.5) | 1 | 1 | (−31.6, −3.1) | (8.9, 27.8) | (16.0, 56.5) |

Evaluated on the subset with all PPG morphology data available

| | | | | | | | |
|---|---|---|---|---|---|---|---|
| Metadata + | 71.7 | −1.5 | <0.01 | 1 | −1.5 | −0.2 | 1.3 |
| PPG | (71.3, 72.0) | (−2.9, −0.1) | | | (−3.7, 0.7) | (−2.4, 2.0) | (1.1, 1.6) |
| morphology | | | | | | | |

| | | |
|---|---|---|
| Office-based | 73.1 | n/a (subset reference) |

| | |
|---|---|
| refit-WHO | (72.8, 73.5) |

Evaluated on the subset with laboratory data available

| | | | | | | | |
|---|---|---|---|---|---|---|---|
| Lab-based | 74.8 | 1.5 | <0.01 | <0.01 | 2.7 | 4.3 | 1.5 |
| refit-WHO | (74.4, 75.2) | (0.1, 2.5) | | | (1.1, 4.4) | (2.6, 6.0) | (1.3, 1.8) |

| | | |
|---|---|---|
| Office-based | 73.4 | n/a (subset reference) |

| | |
|---|---|
| refit-WHO | (73.0, 73.8) |

### VI. Conclusion

The particular arrangements shown in the Figures should not be viewed as limiting. It should be understood that other embodiments may include more or less of each element shown in a given Figure. Further, some of the illustrated elements may be combined or omitted. Yet further, an exemplary embodiment may include elements that are not illustrated in the Figures.

Additionally, while various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are contemplated herein.

1. A method for training a composite model to predict cardiovascular disease risk, the method comprising:
training a machine learning model of the composite model to output, based on an input photoplethysmographic waveform, a first set of features that are representative of the photoplethysmographic waveform;
determining, based on the photoplethysmographic waveform, a heart rate;
determining, based on the first set of features, a mapping of the composite model to project the first set of features to a second set of features, wherein the second set of features includes fewer features than the first set of features;
determining a set of model coefficients of the composite model to predict, based on the second set of features, the heart rate, and a set of demographic information, a cardiovascular disease risk score; and
outputting an indication of the machine learning model, mapping, and set of model coefficients of the composite model.

2. The method of claim 1, wherein the second set of features is a set of five features.

3. The method of claim 1, wherein the set of demographic information for the subject includes at least one of an age of the subject, a sex of the subject, and a smoking status of the subject.

4-6. (canceled)

7. The method of claim 1, wherein training the machine learning model comprises training the machine learning model in combination with a multi-task output model that receives, as input, feature vectors generated by the machine learning model and that generates, as output, sets of measured physiological data corresponding to the individuals from whom photoplethysmographic waveforms input into the machine learning model during training were taken.

8. The method of claim 7, wherein the sets of measured physiological data predicted by the output model include sex, age, BMI, hypertension status, hba1c, total cholesterol, systolic blood pressure, previous experience of at least one previous major adverse cardiovascular event, and PPG dicrotic notch presence.

9. The method of claim 1, wherein training the machine learning model comprises training the machine learning model using an augmented training dataset that includes the input photoplethysmographic waveform and a second photoplethysmographic waveform, wherein the second photoplethysmographic waveform has been generated by:
generating a random time-varying time shift; and
applying the time-varying time shift to the input photoplethysmographic waveform to generate the second photoplethysmographic waveform.

10. The method of claim 1, wherein the set of model coefficients are a set of coefficients that define a Cox proportional hazard model.

11. The method of claim 1, wherein determining the mapping to project the first set of features to the second set of features comprises determining a set of principal component analysis (PCA) eigenvectors of the composite model to linearly project the first set of features to the second set of features, and wherein outputting the indication of the mapping comprises outputting an indication of the set of PCA eigenvectors.

12. A method comprising:
obtaining a photoplethysmographic waveform for a subject;
determining, based on the photoplethysmographic waveform, a heart rate for the subject;
applying the photoplethysmographic waveform to a machine learning model to generate a set of features for the subject; and
applying the heart rate, the set of features, and a set of demographic information for the subject to a statistical model to determine a cardiovascular disease risk for the subject.

13. The method of claim 12, wherein generating the set of features for the subject comprises:
applying the photoplethysmographic waveform to a machine learning model to generate a feature vector; and
applying the feature vector to a linear embedding matrix to project the feature vector into a lower-dimensional linear embedding space, thereby generating the set of features.

14. The method of claim 13, wherein the embedding matrix is a matrix of principal component analysis (PCA) eigenvectors.

15. The method of claim 14, wherein the embedding matrix is a matrix of five PCA eigenvectors.

16. The method of claim 12, wherein the set of demographic information for the subject includes an age of the subject, a sex of the subject, and a smoking status of the subject.

17. The method of claim 12, wherein the machine learning model generates, from the input photoplethysmographic waveform, a vector of at least 512 features.

18. The method of claim 12, wherein the machine learning model comprises a deep learning model having a ResNet18 architecture.

19. (canceled)

20. The method of claim 12, wherein the machine learning model has been trained in combination with a multi-task output model that receives, as input, feature vectors generated by the machine learning model and that generates, as output, sets of measured physiological data corresponding to the individuals from whom photoplethysmographic waveforms input into the machine learning model during training were taken.

21. The method of claim 20, wherein the sets of properties of photoplethysmographic waveforms predicted by the output model include sex, age, BMI, hypertension status, hba1c, total cholesterol, systolic blood pressure, previous experience of at least one previous major adverse cardiovascular event, and PPG dicrotic notch presence.

22. (canceled)

23. The method of claim 12, wherein the statistical model is a Cox proportional hazard model.

24. The method of claim 12, further comprising at least one of (i) providing an indication of the determined cardiovascular disease risk on a display, (ii) based on the determined cardiovascular disease risk, determining a likelihood of hospitalization related to cardiovascular disease, (iii) based on the determined cardiovascular disease risk, determining an aggregate risk score across a number of diseases or syndromes related to cardiovascular disease, (iv) based on the determined cardiovascular disease risk, determining a likelihood that the subject will need to take a specified drug or receive a specified treatment, (iv) responsive to determining that the determined cardiovascular disease risk exceeds a threshold risk level, providing a treatment to the subject and/or performing an additional diagnostic on the subject.

25-27. (canceled)

28. A non-transitory computer readable medium having stored thereon program instructions executable by at least one processor to cause the at least one processor to perform operations compromising:
obtaining a photoplethysmographic waveform for a subject;
determining, based on the photoplethysmographic waveform, a heart rate for the subject;
applying the photoplethysmographic waveform to a machine learning model to generate a set of features for the subject; and
applying the heart rate, the set of features, and a set of demographic information for the subject to a statistical model to determine a cardiovascular disease risk for the subject.

29. (canceled)
